(19) United States
(12) Patent Application Publication
(10) Pub. No.: US 2019/0082929 A1
(43) Pub. Date: Mar. 21, 2019

(54) ENDOSCOPE MANAGEMENT SYSTEM, ENDOSCOPE DEVICE, MANAGEMENT DEVICE FOR MANAGING ENDOSCOPE DEVICE, AND METHOD FOR MANAGING ENDOSCOPE DEVICE

(71) Applicant: HOYA CORPORATION, Tokyo (JP)

(72) Inventor: Hiroyuki WATANABE, Tokyo (JP)

(73) Assignee: HOYA CORPORATION, Tokyo (JP)

(21) Appl. No.: 16/084,087

(22) PCT Filed: Apr. 27, 2017
(86) PCT No.: PCT/JP2017/016755

§ 371 Date: Sep. 11, 2018

(30) Foreign Application Priority Data
Apr. 27, 2016 (JP) 2016-089480

(51) Int. Cl. A61B 1/00; A61B 1/045; A61B 1/05; A61B 1/06

(52) U.S. Cl. CPC A61B 1/00006; A61B 1/045; A61B 1/00009; A61B 1/00163; A61B 1/06; A61B 1/00096; A61B 1/05

(57) ABSTRACT

An endoscope management system that manages deterioration of an endoscope device includes: at least one endoscope device; and a management device configured to be connected to a processor of the endoscope device such that the management device is able to perform mutual communication with the processor. The endoscope device includes a video scope and a processor, the video scope being configured to capture an image of a subject, and the processor including an image signal processing unit configured to execute white balance adjustment processing, using an image of the subject captured by the video scope. The endoscope device includes a control unit configured to transmit, in response to the execution of the white balance adjustment processing, monitoring-target subject images acquired through the white balance adjustment processing, to the management device. The management device includes a memory configured to record the monitoring-target subject images, and a main management unit.

### TECHNICAL FIELD

The present invention relates to an endoscope management system for an endoscope such as a video scope, an endoscope device, a management device that manages an endoscope device, and a method for managing an endoscope device, and particularly relates to an endoscope management system and a method for managing an endoscope device for centrally managing a plurality of endoscope devices.

### BACKGROUND ART

An endoscope management system is provided with a management system that performs mutual communication with each of a plurality of endoscope devices that are installed in a hospital or the like, and the management system performs filing of data regarding usage of the video scope and the processor of each of the endoscope devices, and monitors the data. For example, there is a well-known endoscope management system in which a management device receives data regarding cumulative usage time (e.g. usage start time and end time) of a video scope, a processor, and a lamp, and detects cumulative usage time or the like of each device. In this endoscope management system, the management device transmits the detected information to an endoscope device so that a monitor of the endoscope device displays the information (see Patent Document 1).

There is also a well-known endoscope management system in which an endoscope management device in the endoscope management system records the number of bending operations that have been performed to bend a scope bending part of an endoscope device, and upon determining that the total number of bending operations has reached a certain number indicating that maintenance is necessary, the endoscope management device transmits a warning command to the endoscope device so that a warning is displayed on a monitor (see Patent Document 2).

### PRIOR ART DOCUMENTS

### Patent Documents

Patent Document 1: JP 2012-147959A

Patent Document 2: JP 2012-245254A

### SUMMARY OF INVENTION

### Problem to be Solved by Invention

In some cases, an abnormality may occur in an observation image observed by an endoscope device, due to a signal cable in the video scope deteriorating as a result of being used. However, even if the scope cumulative usage time or the like is transmitted to the management system, the management device in the management system cannot ascertain whether or not an abnormality is present in the observation image.

In addition, the task of regularly transmitting observation images for monitoring to the management device in the management system is burdensome for operators, and it is often the case that an operator forgets to transmit an observation image. Also, monitoring-target subject images, contained in images recorded by a filing device in the management device, differ from each other, and image quality such as color tone is different for each subject image. Therefore, it is difficult to fixedly use uniform monitoring-target subject images.

In particular, a video scope of an endoscope device is provided with a flexible thin cable and a small leading end part that is provided at the leading end of the cable and includes an imaging element, an optical system, and so on, and has an insertion part that is to be inserted into a human body, and, in some cases, inserted into a human body while being in contact with living tissues in the human body. Therefore, the cable and the leading end part inevitably deteriorate as a result of the use of the video scope. Therefore, it is important to manage the deterioration of the video scope.

Thus, the management device is required to acquire uniform monitoring-target subject images, without an operator performing a special operation, and the management device is required to accurately ascertain deterioration of a device, which is an endoscope device.

### Means for Solving Problem

One embodiment of the present invention is an endoscope management system. The endoscope management system includes:

at least one endoscope device that includes a video scope and a processor, the video scope being configured to capture an image of a subject, and the processor including an image signal processing unit configured to execute white balance adjustment processing, using an image of the subject captured by the video scope; and

a management device configured to be connected to the processor of the endoscope device such that the management device is able to perform mutual communication with the processor.

The endoscope device includes a control unit configured to transmit, in response to the execution of the white balance adjustment processing, monitoring-target subject images acquired through the white balance adjustment processing, to the management device, and

The management device includes a memory configured to record the monitoring-target subject images, and a main management unit configured to manage deterioration of the endoscope device, using the monitoring-target subject images thus recorded.

Another embodiment of the present invention is also an endoscope management system. The endoscope management system includes at least one endoscope device that includes a white balance adjustment processing unit that executes white balance adjustment processing according to an operation performed by an operator; and

a management device that is connected to the endoscope device such that the management device is able to perform mutual communication with the endoscope device.

The endoscope device transmits, in response to the execution of the white balance adjustment processing, monitoring-target subject images acquired through the white balance adjustment processing, to the management device, and

the management device records the monitoring-target subject images thus received, in a memory.

It is preferable that the endoscope device includes a video scope and a processor, the video scope being configured to capture an image of a subject, and the processor including an image signal processing unit including the white balance adjustment processing unit, the white balance adjustment processing unit being configured to execute white balance adjustment processing, using images of the subject captured by the video scope as the monitoring-target subject images.

The monitoring-target subject images to be transmitted to the management device are images that are acquired at the time of white balance adjustment (hereinafter referred to as “images at the time of white balance adjustment”). The images at the time of white balance adjustment are usually images of the tubular inner surface of a white balance adjustment tool. Therefore, for example, still color images of the tubular inner surface, which have undergone white balance adjustment processing, are transmitted as monitoring-target subject images. Here, “at the time of white balance adjustment processing” may be a point in time after white balance adjustment processing has been started, and before the completion of white balance adjustment processing, immediately after the completion of adjustment processing, or when a predetermined period of time has elapsed after the completion. Considering that it is difficult to hold the leading end part of the video scope in the tube, which is the adjustment tool, for a long time, for example, it is preferable that “at the time of white balance adjustment processing” is a point in time immediately after the completion of white balance adjustment processing. Therefore, it is preferable that the endoscope device transmits monitoring-target subject images at a point in time immediately after the completion of white balance adjustment processing, to the management device.

It is preferable that the endoscope device transmits scope-related information related to the video scope of the endoscope device, in conjunction with transmission of the monitoring-target subject images.

It is preferable that the scope-related information includes information regarding a usage history of the video scope and identification information of the video scope.

It is preferable that information regarding the usage history of the video scope includes cumulative scope-energization time that indicates cumulative time for which the video scope has been energized, or scope connection count that indicates a number of times the video scope has been connected to the processor.

It is preferable that the endoscope device transmits processor-related information related to the processor of the endoscope device, in conjunction with transmission of the monitoring-target subject images.

It is preferable that the processor includes a processing circuit that is able to turn ON and OFF image processing that is to be performed on images of the subject captured by the video scope, and

the processing circuit turns the image processing OFF when the white balance adjustment processing is to be performed.

It is preferable that the endoscope device transmits scope-related information related to the video scope and processor-related information related to the processor to the management device together with the monitoring-target subject images, and

the main management unit associates the plurality of monitoring-target subject images with different dates recorded in the memory, with at least one of the scope-related information and the processor-related information transmitted in conjunction with each of the plurality of monitoring-target subject images, to determine whether or not the endoscope device requires maintenance.

It is also preferable that the scope-related information includes information regarding cumulative scope-energization time that indicates cumulative time for which the video scope has been energized, and

the management device determines that the endoscope device requires maintenance when the cumulative scope-energization time is no less than a predetermined period of time.

It is preferable that the management device

determines whether or not the endoscope device requires maintenance based on image analysis performed on the monitoring-target subject images recorded in the memory.

It is preferable that the management device determines that the endoscope requires maintenance when a color tone of any of the monitoring-target subject images is out of an allowable range. For example, it is possible to make a determination based on the ratio between R (red), G (green), and B (blue) color image signals in the monitoring-target subject images.

It is preferable that the management device notifies the endoscope device of the result of the determination regarding whether or not the endoscope device requires maintenance.

It is preferable that the management device is connected to a monitor that is controlled such that the monitor displays the monitoring-target subject images together with at least one of scope-related information related to the video scope of the endoscope device and processor-related information related to the processor.

Another aspect of the present invention is an endoscope device. The endoscope device includes:

a video scope that captures an image of a subject; and

a processor including an image signal processing unit and a control unit, the image signal processing unit executing white balance adjustment processing, using images of the subject captured by the video scope, and the control unit transmitting, in response to the execution of the white balance adjustment processing, monitoring-target subject images acquired through the white balance adjustment processing, to a management device, which is an external device.

Another aspect of the present invention is a management device that is to be connected to the endoscope device such that the management device is able to perform mutual communication with the endoscope device, the management device including: a communication unit that receives the monitoring-target subject images output from the endoscope device; a memory that records the monitoring-target subject images thus received; and a main management unit that manages deterioration of the endoscope device, using the monitoring-target subject images thus recorded.

Another aspect of the present invention is a method for managing an endoscope device. According to the management method, at least one endoscope device that includes an image signal processing unit configured to execute white balance adjustment processing is connected to a management device such that the endoscope device is able to perform mutual communication with the management device.

(1) The endoscope device transmits, in response to the execution of the white balance adjustment processing, a monitoring-target subject image acquired through the white balance adjustment processing, to the management device;

(2) the management device records the monitoring-target subject images thus received, in a memory; and

(3) the management device manages the deterioration of the endoscope device, using the monitoring-target subject images thus recorded.

### Advantageous Effects of Invention

With the endoscope management system, the endoscope device, the management device that manages the endoscope device, and the method for managing the endoscope device above, the management device can accurately ascertain deterioration of the endoscope device.

### BRIEF DESCRIPTION OF DRAWINGS

FIG. 1 is a block diagram showing an example of an electronic endoscope device according to a first embodiment.

FIG. 2 is a flowchart showing an example of a series of white balance adjustment processing and monitoring-target subject image transmission processing that is executed in a processor of the electronic endoscope device according to the first embodiment.

FIG. 3 is a diagram showing an example of a monitoring-target subject image.

FIG. 4 is a flowchart showing an example of filing that is executed in a controller included in a management device according to the first embodiment.

FIG. 5 is a diagram showing an example of information in the form of a database created by the management device according to the first embodiment.

FIG. 6 is a flowchart showing an example of a series of filing and analyzing processing that is executed in a management device according to a second embodiment.

FIG. 7 is a diagram chronologically showing examples of monitoring-target subject images received by the management device according to the second embodiment.

FIG. 8 is a flowchart showing an example of maintenance warning processing that is executed in an endoscope device of the management device according to the second embodiment.

FIG. 9 is a flowchart showing an example of a series of filing and analyzing processing that is executed in a management device according to a third embodiment.

FIG. 10 is a diagram showing examples of monitoring-target subject images received by the management device according to the third embodiment.

### DESCRIPTION OF EMBODIMENTS

The following describes an endoscope management system, an endoscope device, a management device that manages an endoscope device, and a method for managing an endoscope device according to embodiments with reference to drawings.

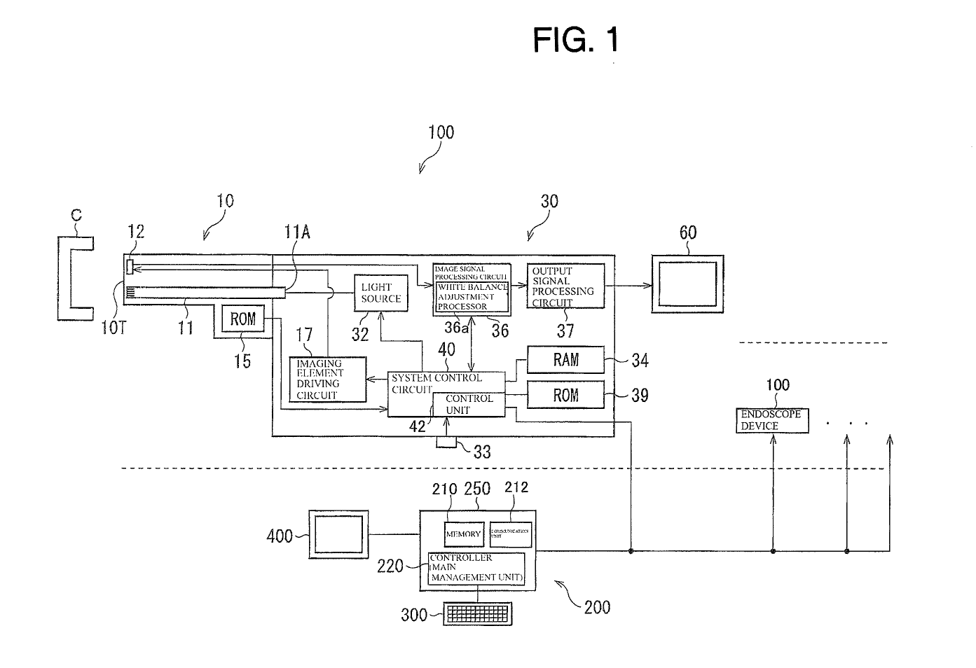

FIG. 1 is a block diagram showing an endoscope management system according to a first embodiment.

The endoscope management system includes a plurality of endoscope devices 100 that each includes a video scope 10 that is to be inserted into a body, and a processor 30. Each endoscope device 100 is connected to, and is able to communicate with, a management device (hereinafter also referred to as a “filing device”) 200. The endoscope management system can be constructed in a hospital, for example, and endoscope devices 100 of different types or the same type are installed in laboratories and operating rooms. The filing device 200 is installed in a central management room or the like. A video scope 10 of each endoscope device 100 is detachably connected to a processor 30, and a monitor 60 is connected to the processor 30. Therefore, the plurality of video scopes 10 can be freely connected to a plurality of processors 30. The video scope 10 includes a flexible thin cable and a small leading end part that is provided at the leading end of the cable and includes an imaging element, an optical system, and so on. It is likely that deterioration of the cable and the leading end part progresses as a result of the use of the video scope. Therefore, it is preferable that deterioration of the video scopes 10 is managed.

The processor 30 includes a light source 32 such as a xenon lamp, which is lit up upon a power button (not shown) being operated. Light emitted from the light source 32 enters an incident end 11A of a light guide 11 that is provided in the video scope 10, via a light collection lens (not shown). Light emitted from the light guide 11 is emitted from a scope leading end part 10T to a subject (an observation target) via a light distribution lens (not shown). A diaphragm (not shown) is provided between the light source 32 and the light guide 11, and the amount of illumination light is adjusted by opening and closing the diaphragm.

Illumination light that is reflected off the subject is formed into an image by an objective lens (not shown) provided at the scope leading end part 10T, and thus a subject image is formed on a light-receiving surface of an image sensor 12. The image sensor 12, which is constituted by CMOS imaging elements or CCD imaging elements, is driven by an imaging element driving circuit 17, and image signals of one field or one frame are read out from the image sensor 12 at a predetermined frame rate (e.g. at intervals of 1/60 seconds or 1/30 seconds). A color filter array (not shown), which is an array of color filters of Cy (cyan), Ye (yellow), G (green), and Mg (magenta), or R (red), G (green), and B (blue), for example, is provided on the light-receiving surface of the image sensor 12.

Image signals read out from the image sensor 12 are digitized in an initialization circuit (not shown), and are thereafter transmitted to an image signal processing circuit 36 in the processor 30. In the image signal processing circuit 36, image processing, such as color conversion processing and gamma correction processing, is performed on digital image signals. As a result, R, G, and B color image signals are generated. Furthermore, the image signal processing circuit (image signal processing unit) 36 includes a white balance adjustment processor 36a, which performs white balance adjustment processing to adjust the gain values of R, G, and B color image signals.

R, G, and B image signals are temporarily stored in an image memory (not shown) such as a RAM, and are thereafter transmitted to an output signal processing circuit 37. The output signal processing circuit 37 performs, for example, image processing such as contour enhancement processing, which is performed in response to an operation input by an operator, superimposing processing, which is performed when text information is input, and so on, on image signals. Video signals are output to the monitor 60, and thus an observation image is displayed on the monitor 60.

Note that, according to an embodiment, a processing circuit that includes the image signal processing circuit 36 and the output signal processing circuit 37 is configured to be able to switch ON or OFF the image processing that is to be performed on the subject image captured by the video scope 10. This processing circuit is configured to turns image processing OFF after white balance adjustment processing has been executed.

A system control circuit 40 that includes a CPU and so on outputs a control signal to a timing generator (not shown), the image signal processing circuit 36, and so on, and controls the operation of the processor 30 while the processor 30 is in a power-on state. An operation control program is stored in a ROM 39 in advance. In addition, the system control circuit 40 includes a control unit 42 that transmits images and various kinds of information to the management device 200, which will be described later, and furthermore, receives information from the management device 200.

Upon the video scope 10 being connected to the processor 30, the system control circuit 40 reads out scope-related information stored in a ROM 15 of the video scope 10, and stores the information in a RAM 34.

Scope-related information includes at least information regarding the usage history of the video scope 10 and the serial number (identification information) of the video scope 10. According to an embodiment, scope-related information includes pieces of information including the model name of the video scope 10, the serial number of the video scope 10, the characteristics (e.g. the number of pixels) of the image sensor 12, a cumulative scope-energization time and a scope connection count of the video scope 10, and so on. The cumulative scope-energization time is the cumulative time for which the video scope 10 has been energized. The scope connection count is the number of times the video scope 10 has been connected to a specific processor 30. Information regarding the usage history of the video scope 10 includes the cumulative scope-energization time and the scope connection count of the video scope 10.

The endoscope device 100 can execute white balance adjustment processing. The operator presses a white balance button 33, with the leading end part 10T of the video scope 10 being inserted into a white balance adjustment tool C, which is tubular. As a result, the image signal processing circuit 36 executes white balance adjustment processing. The gain values of the R, G, and B color image signals are adjusted based on an image of the white tubular inner surface of the white balance adjustment tool C such that the ratio between the values of the R, G, and B color image signals is 1:1:1. After white balance adjustment processing has been performed, color image signals are generated based on the gain values thus set.

When the video scope 10 is to be removed from the processor 30, the system control circuit 40 detects the usage time (hereinafter referred to as “energization time”) of the connected video scope 10 in association with the scope model name and the serial number, adds the period of time from the scope's connection (ON) to the scope's removal (OFF) to the cumulative scope-energization time stored in the ROM 15 of the video scope 10, and thus rewrites the cumulative scope-energization time stored in the ROM 15. Upon the video scope 10 being connected to the processor 30, the system control circuit 40 increments the connection count by one in association with the scope model name and serial number, and thus rewrites the connection count stored in the ROM 15. Furthermore, the system control circuit 40 detects the usage time of the light source 32, which is the period of time from ON to OFF of the power button, updates light source cumulative usage time, and records it in the ROM 39.

On the other hand, the filing device (management device) 200 installed on the management side is constituted by a computer 250, for example. A keyboard 300 and a monitor 400 are connected to the computer 250. The filing device 200 includes a communication unit 212, which is configured to be connected to the system control circuit 40 of the endoscope device 100 such that it is able to perform mutual data communication, a controller 220, and a memory 210.

The memory 210 is configured to record subject images for monitoring, which are transmitted from the processor 30, scope-related information, and processor-related information.

The controller 220 includes a CPU (Central Processing Unit) of the computer 250 and is configured to manage deterioration of the endoscope device 100, using the recorded subject images for monitoring. That is, the controller 220 is a main management unit that is configured to manage deterioration of the endoscope device 100.

The communication unit 212 of the filing device 200 transmits predetermined information such as command data to the endoscope device 100 as necessary. The system control circuit 40 of the endoscope device 100 transmits data to the filing device 200 via the control unit 42 as necessary.

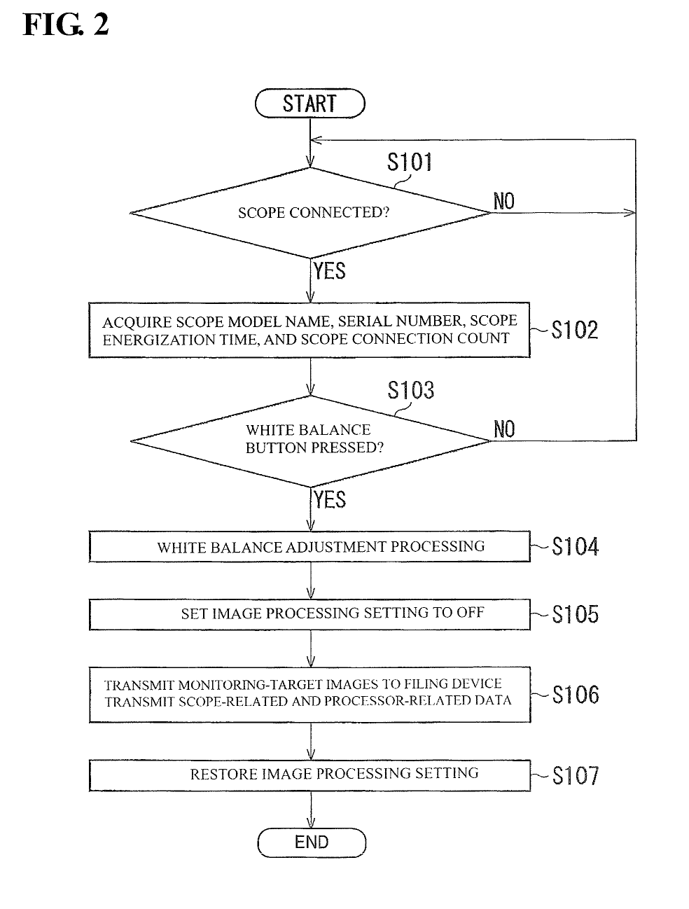

In the present embodiment, after white balance adjustment processing has been performed, the control unit 42 transmits a subject image that has undergone white balance adjustment (hereinafter referred to as a “monitoring-target subject image”), and in conjunction with this transmission, transmits processor-related information including scope-related information and light source-related information to the filing device (management device) 200 on the management side. Processor-related information includes, for example, the model name of the processor 30, the serial number of the processor 30, and the operation time of the processor 30. Light source-related information includes, for example, the cumulative usage time of the light source 32. The following describes management of endoscope devices 100 in detail with reference to FIGS. 2 to 5.

In such an endoscope management system, at least one endoscope device 100 that includes the image signal processing circuit (image signal processing unit) 36 that is configured to execute white balance adjustment processing is connected to the management device 200 such that the endoscope device 100 is able to perform mutual communication with the management device 200.

In response to white balance adjustment processing being performed, the endoscope device 100 transmits a monitoring-target subject image IM, which is acquired at the time of white balance adjustment processing, to the management device 200.

The management device 200 records the received monitoring-target subject images IM in the memory 210.

Furthermore, the management device 200 manages deterioration of the endoscope device 100, using the recorded monitoring-target subject images IM.

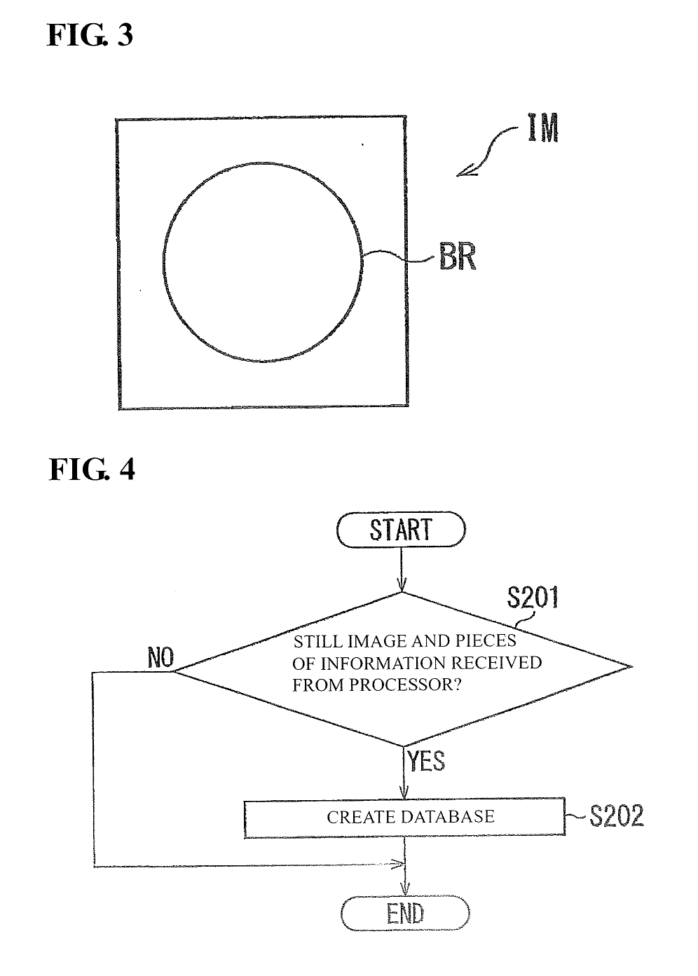

FIG. 2 is a flowchart showing an example of a series of white balance adjustment processing and monitoring-target subject image transmission processing that is executed by the processor 30. The flow shown in FIG. 2 starts upon the processor 30 being powered ON. FIG. 3 is a diagram showing an example of a monitoring-target subject image.

Upon the processor 30 determining that the video scope 10 is connected thereto, scope-related information, i.e., information regarding the scope model name, the serial number, the scope-energization time (the cumulative scope-energization time), characteristics of the image sensor, and the scope connection count are read out from the ROM 15 of the video scope 10 (S101 and S102).

Upon the processor 30 determining that the white balance button 33 has been pressed by the operator, white balance adjustment processing is executed, and the gain values of the R, G, and B color image signals of the monitoring-target subject image, which is an image of the white tubular inner surface of the white balance adjustment tool C, are adjusted so that the ratio between the R, G, and B color image signals is 1:1:1 (S103 and S104).

After white balance adjustment processing has been executed, the system control circuit 40 turns OFF image processing that has been set in the processor 30 (S105). For example, if contour enhancement processing has been set to ON, the system control circuit 40 turns OFF the processing.

Thereafter, a still color image that has undergone white balance adjustment processing (gain value adjustment) is transmitted from the control unit 42 to the filing device 200 as a monitoring-target subject image (S106). As shown in FIG. 3, the monitoring-target subject image IM is an image of the tubular inner surface of the white balance adjustment tool C. The scope leading end part 10T is guided to an insertion position, and therefore a contour BR of the bottom circle of the tube is displayed at substantially the same position in the screen regardless of when the monitoring-target IM is acquired.

Also, scope-related information including information regarding the model name of the video scope 10 connected to the processor 30, the serial number of the video scope 10, cumulative scope-energization time, and the scope connection count, and processor-related information related to the processor 30 (the model name of the processor 30, the serial number of the processor 30, the processor's operation time, and the light source cumulative usage time) are transmitted to the filing device 200 in conjunction with transmission of the monitoring-target subject image IM (S106). Upon the monitoring-target subject image IM, scope-related information, and processor-related information being transmitted, the system control circuit 40 turns ON the image processing that has been set to OFF (e.g. turns ON contour enhancement) (S107). Thus, white balance adjustment processing and monitoring-target subject image transmission processing end.

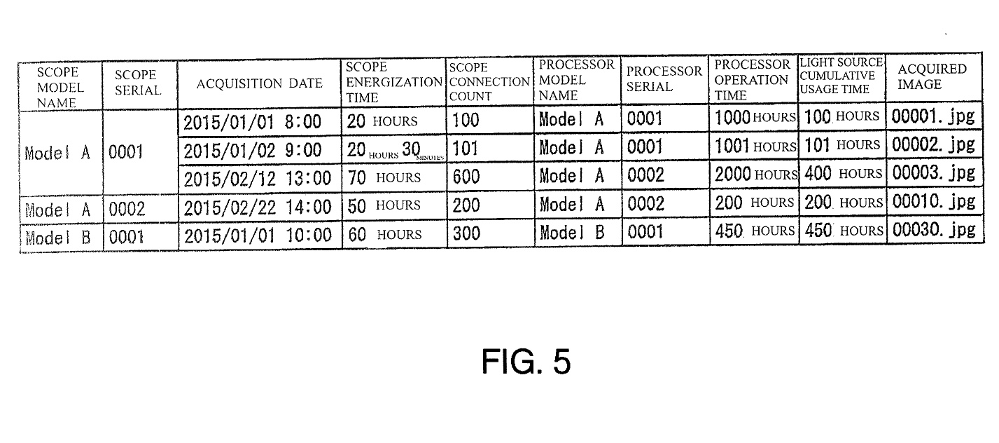

FIG. 4 is a flowchart showing an example of filing that is executed by the controller 220 of the filing device 200. FIG. 5 is a diagram showing information in the form of a database. Here, the filing processing shown in FIG. 4 is executed as interrupt processing that interrupts the main routine shown in FIG. 2.

In step S201, the filing device 200 determines whether or not the filing device 200 has received a monitoring-target subject image IM, scope-related information, and processor-related information from any of the endoscope devices that are connected to the filing device 200. Upon determining that the aforementioned pieces of information have been received, the filing device 200 creates a database from them (S202). Specifically, the filing device 200 records the monitoring-target subject image IM in the memory 210 in association with the scope model name and the scope serial number (the identification information of the scope). At this time, the filing device 200 also records the scope-energization time (the cumulative scope-energization time), the scope connection count, the light source cumulative usage time, the processor model name, the processor serial number (the identification information of the processor) and so on, in association with the scope model name and the scope serial number at the same time.

FIG. 5 shows part of a database regarding monitoring-target subject images IM associated with scope-related information and processor related information that includes light source-related information.

Monitoring-target subject images IM are recorded in association with each combination of a scope model name and a scope serial number. A monitoring-target subject image IM is displayed on the monitor 400 upon the operator operating the keyboard 300. Also, scope-related information such as the scope model name, the scope serial number, the scope-energization time (the cumulative scope-energization time), and the scope connection count, and processor-related information including light source-related information are displayed together with the monitoring-target subject image IM. That is, the filing device (management device) 200 is connected to the monitor 400, and the monitor 400 is controlled by the controller (main management unit) 220 such that it displays a monitoring-target subject image IM together with at least one of scope-related information and processor-related information.

Thus, the controller 220 can examine deterioration of a device, which is the video scope 10 or the processor 30, based on a monitoring-target subject image IM, a scope-related information, and processor-related information including light source-related information displayed on the monitor 400. According to one embodiment, it is preferable that the main management unit 220 analyzes the monitoring-target subject image IM and examines noise in the image or the color tone of the monitoring-target subject image IM to determine deterioration of the endoscope device 100. If this is the case, it is preferable that the main management unit 220 associates the monitoring-target subject image IM, scope-related information, and processor-related information including light source-related information with each other to determine deterioration of the endoscope device 100. For example, since scope-related information and processor-related information include information regarding the usage history of the video scope 10 and the usage history of the light source (the light source cumulative usage time) it is possible to associate these pieces of information with the results of the examination of the monitoring-target subject image IM (e.g. noise and an abnormal color tone) to reliably determine deterioration of the endoscope device 100.

In particular, since monitoring-target subject images IM are chronologically recorded for the same scope model name and scope serial number and the same processor model name and processor serial number, it is possible to perform an image comparison to determine the degree of deterioration in the monitoring-target subject images IM to determine deterioration of the endoscope device 100. The aforementioned image comparison is a comparison between the most recently acquired monitoring-target subject image IM and a monitoring-target subject image that has been acquired before the most recently acquired monitoring-target subject image IM. A monitoring-target subject image that has been acquired before the most recently acquired monitoring-target subject image IM is, for example, an monitoring-target subject image IM of which the acquisition date is the closest to that of the most recently acquired monitoring-target subject image IM, a monitoring-target subject image IM that has been acquired on a date at a predetermined interval from the acquisition date of the most recently acquired monitoring-target subject image IM, or a monitoring-target subject image IM that has been acquired in an early stage of the use of the endoscope device.

In addition, the filing device 200 also performs filing of monitoring-target subject images IM that, despite corresponding to the same scope model name and scope serial number, correspond to combinations of different processor model names and processor serial numbers, and performs filing of monitoring-target subject images IM that, despite corresponding to different scope model names and scope serial numbers, correspond to combinations of the same processor model name and processor serial number. Therefore, according to an embodiment, the main management unit 220 can determine which of the video scope 10 and the processor 30 has deteriorated by examining a plurality of monitoring-target subject images IM. Furthermore, according to an embodiment, it is possible to predict and determine deterioration of a device, which is a video scope 10 or a processor 30, by associating monitoring-target subject images IM and the scope-energization time (the cumulative scope-energization time) or information regarding the scope connection count with each other.

For example, if the filing device 200 determines that a device has deteriorated based on a plurality of monitoring-target subject images IM that are chronologically displayed, the filing device 200 transmits a command indicating that the device has deteriorated, to the operator who is working using the device, and prompts the operator to replace the device. Also, if the filing device 200 has successfully extracted the time at which the device will deteriorate (based on the cumulative energization time) by performing the aforementioned analysis, the filing device 200 can transmit, to the subject endoscope device, a command indicating that the device is expected to deteriorate.

That is, the controller 220 associates each of a plurality of monitoring-target subject images in the memory 210 acquired at different dates, and at least one of the scope-related information and the processor-related information that have been transmitted together with the monitoring-target subject image, with each other, to predict deterioration of a device, which is the video scope 10 or the processor 30, and determine whether or not it is necessary to perform maintenance on the endoscope device 100.

In this way, according to the present embodiment, in an endoscope management system that includes a plurality of endoscope devices 100 and the filing device 200 that can perform mutual communication, an endoscope device 100, upon performing white balance adjustment processing, transmits a monitoring-target subject image IM that has undergone white balance adjustment processing to the filing device 200. Also, scope-related information and processor-related information are transmitted at the same time.

Since monitoring-target subject images IM can be regularly transmitted to the filing device 200 without an operator performing a special operation, the filing device 200 on the management side can accurately determine whether or not the endoscope device 100 has deteriorated. Also, since white balance adjustment processing is usually performed while the processor is powered ON, monitoring-target subject images IM are acquired at appropriate time intervals. Furthermore, since monitoring-target subject images are images of the same subject, which is the inner surface of a tube, whether or not an image shows deterioration can be accurately determined with reference to a monitoring-target subject image IM that has been transmitted in an early stage of usage. Then, a monitoring-target subject image IM is transmitted after image processing, such as contour enhancement, has been set to OFF. Therefore, the filing device 200 can record a monitoring-target subject image IM that has natural contrast and appropriate brightness.

The filing device 200 files monitoring-target subject images IM in association with each of the scope model names and scope serial numbers of the video scopes 10 connected to the filing device 200. Therefore, the filing device 200 compares monitoring-target subject images IM that have a color tone corresponding to the image sensor or the fiber characteristics of a video scope 10 connected the filing device 200. Thus, for example, the filing device 200 compares the most recently acquired monitoring-target subject image IM with a monitoring-target subject image that has been acquired before the most recently acquired monitoring-target subject image IM. Therefore, even if the usage history shows that various video scopes have been connected to the processor 30, it is possible to determine whether or not the image is abnormal. Furthermore, filing is performed in association with the processor model names and the processor serial numbers, and therefore, even if video scopes and processors of various models are connected, it is possible to accurately analyze chronological changes in the monitoring-target subject images IM with respect to the same scope model name and scope serial number and the same processor model name and processor serial number.

Note that, in the description above, image processing is set to OFF when a monitoring-target subject image IM is to be created. However, image processing may not be set to OFF and may be kept ON when a monitoring-target subject image IM is to be created, and a monitoring-target subject image IM may be created when image processing is ON. If this is the case, it is preferable that the system control circuit 42 transmits information regarding an image processing setting to the filing device 200, and records the setting as part of the database. The above-described analysis performed to determine whether or not a device has deteriorated can be performed considering this image processing setting.

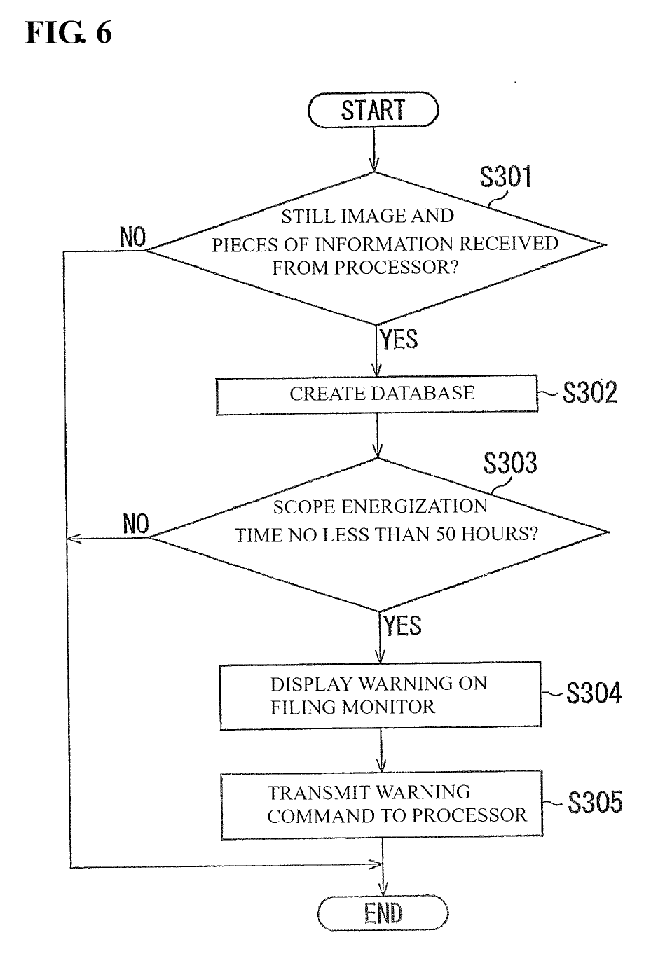

Next, the following describes a second embodiment with reference to FIGS. 6 to 8. In the second embodiment, the controller 220 of the filing device 200 determines whether or not a device has deteriorated based on scope-related information, and upon determining that the device has deteriorated, the controller 220 notifies the target endoscope device 10 of the fact that the device has deteriorated. Other configurations are substantially the same as those in the first embodiment.

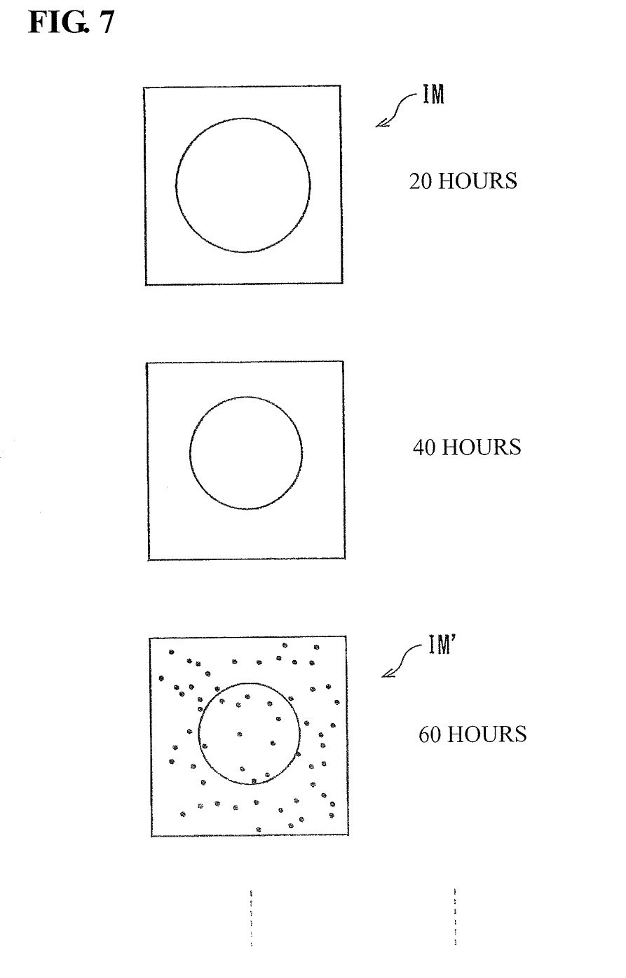

FIG. 6 is a flowchart showing an example of a series of filing and analyzing processing according to the second embodiment. FIG. 7 is a diagram chronologically showing monitoring-target subject images that have been received.

The execution of steps S301 and S302 is the same as the execution of steps S201 and S202 in FIG. 4, and the controller 220 of the filing device 200 records monitoring-target subject images IM, scope-related information, and processor-related information by creating a database. Then, in step S303, the filing device 200 determines whether or not the scope-energization time (the cumulative scope-energization time) is no less than 50 hours.

Here, it is assumed that database analysis performed by the controller 220 has revealed that noise in a monitoring-target subject image IM becomes prominent to a certain extent when the scope-energization time (the cumulative scope-energization time) exceeds 50 hours. The controller 220 sets 50 hours to a threshold value of the scope-energization time (the cumulative scope-energization time) after which deterioration of a device possibly occurs, from a statistical viewpoint. The controller 220 compares this threshold value with the scope-energization time (the cumulative scope-energization time) included in the latest scope-related information in the database corresponding to the scope model name and the scope serial number that are the same as the scope model name and the scope serial number of the scope 10, and performs determination processing to determine whether or not it is necessary to perform maintenance on the endoscope device 100. FIG. 7 shows that a monitoring-target image IM′ when the scope-energization time (cumulative scope-energization time) is 60 hours contains a larger amount of noise than in a monitoring-target image when the scope-energization time (cumulative scope-energization time) is 40 hours, which indicates that the monitoring-target image IM′ has deteriorated.

In step S303, if the controller 220 of the filing device 200 determines that the received scope-energization time (cumulative scope-energization time) with respect to the scope with the same model name and the same scope serial number is no less than 50 hours, the controller 220 performs superimposing processing on the image that is to be displayed in order to display a warning on the monitor 400 (S304). For example, text information saying: “The scope-energization time has exceeded 50 hours. Maintenance should be carried out.” is displayed on the monitor 400. Then, the communication unit 212 of the filing device 200 transmits a warning command indicating that maintenance is necessary, to the target endoscope device 10 (S305).

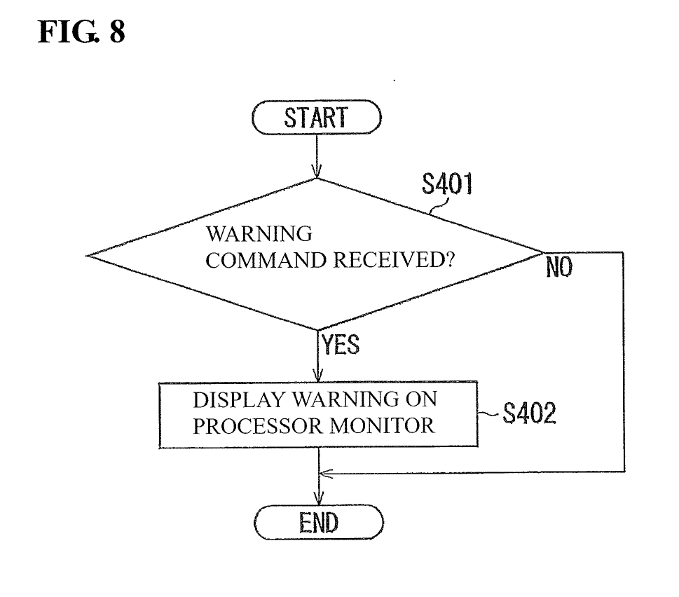

FIG. 8 is a flowchart showing maintenance warning processing that is executed in the endoscope device 10. Here, maintenance warning processing is executed as interrupt processing that interrupts the main routine shown in FIG. 6.

Upon receiving a warning command from the filing device 200, the output signal processing circuit 37 executes superimposing processing to display a warning on the monitor 60 (S401 and S402). For example, text information saying: “The scope-energization time has exceeded 50 hours. Maintenance should be carried out.” is displayed. Note that, if the processor 30 is provided with a buzzer, it is also possible to ring the buzzer at the same time, or only ring the buzzer.

In this way, according to the second embodiment, the controller 220 of the filing device 200 determines whether or not the endoscope device 10 requires maintenance by comparing the threshold value of the scope-energization time (the cumulative scope-energization time) found through analysis performed using the database with the scope-energization time (the cumulative scope-energization time) contained in the latest scope-related information in the database. That is, the controller 220 sets a threshold value of cumulative time, which is used to determine whether or not a device has deteriorated, by using monitoring-target subject images that have been accumulated, and manages deterioration of endoscope devices using this threshold value. Thus, the controller 220 makes it possible to determine whether or not maintenance is necessary, based only on the scope-energization time (the cumulative scope-energization time) contained in scope-related information, without checking the monitoring-target subject images IM. However, in order to accurately determine whether or not the endoscope device 100 requires maintenance, it is preferable to make a comprehensive determination by determine whether or not the monitoring-target subject image IM has deteriorated in addition to making the aforementioned determination using the scope-energization time (the cumulative scope-energization time). For example, if the filing device 200 determines that maintenance is necessary based on the aforementioned determination using the scope-energization time (the cumulative scope-energization time), but no deterioration can be found in the monitoring-target subject image IM, the filing device 200 may determine that the necessity of maintenance on the endoscope device 100 has increased, display this result of determination on the monitor 400, and transmit this result of determination to the endoscope device 100 to display the result of the determination on the monitor 60.

Note that, a relationship between the scope connection count and the deterioration in quality of the monitoring-target subject image may be analyzed instead of the scope-energization time (the cumulative scope-energization time), and whether or not the endoscope device 100 requires maintenance may be determined using the scope connection count. If this is the case, the filing device 200 compares the lower limit of the scope connection count at which maintenance is necessary, with the scope connection count contained in the latest scope-related information in the database corresponding to the scope model name and the scope serial number that are the same as the scope model name and the scope serial number of the scope 10 to determine whether or not the endoscope device 100 requires maintenance. Alternatively, a relationship between the light source cumulative usage time and the deterioration in quality of the monitoring-target subject image IM may be analyzed, and whether or not the endoscope device 100 requires maintenance may be determined using the light source cumulative usage time. If this is the case, the filing device 200 compares the lower limit of the light source cumulative usage time at which maintenance is necessary, with the light source cumulative usage time contained in the latest processor-related information in the database corresponding to the processor model name and the processor serial number that are the same as the processor model name and the processor serial number of the processor 30 to determine whether or not the endoscope device 100 requires maintenance.

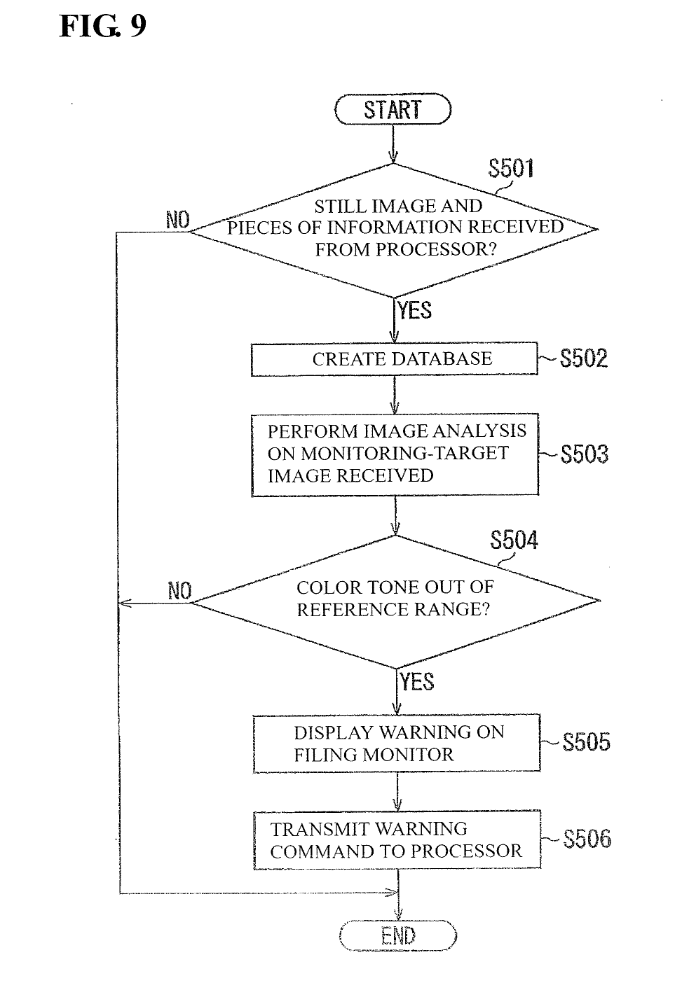

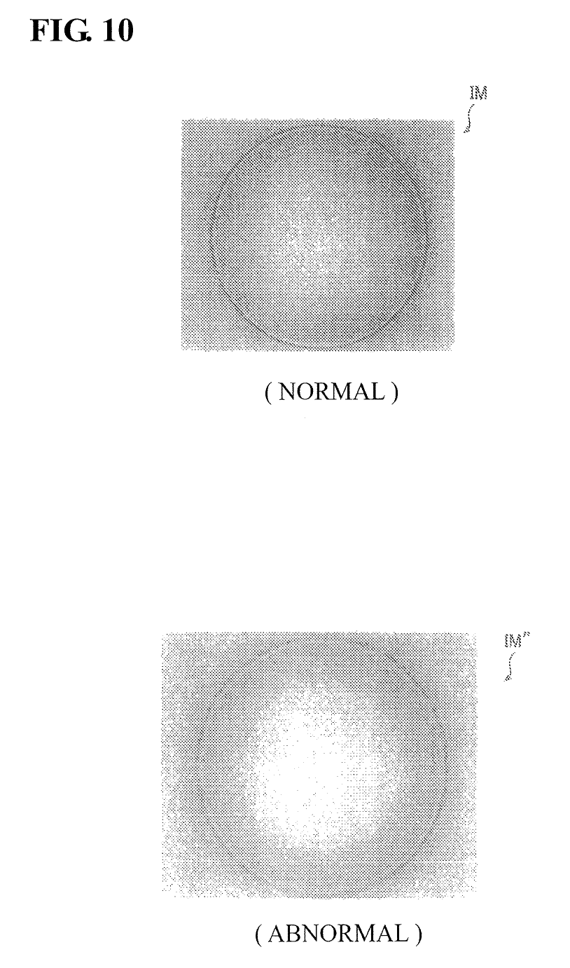

Next, the following describes an endoscope management system, which is a third embodiment, with reference to FIGS. 9 and 10. In the third embodiment, the controller 220 of the filing device 200 determines whether or not the endoscope device 100 has deteriorated by performing image analysis on a monitoring-target subject image IM. Other configurations are substantially the same as those in the first and second embodiments.

FIG. 9 is a flowchart showing an example of a series of filing and analyzing processing according to the third embodiment. FIG. 10 is a diagram showing an example of a monitoring-target subject image IM according to the third embodiment.

The execution of steps S501 and S502 is the same as the execution of steps S201 and S202 in FIG. 4, and the memory 210 of the filing device 200 records monitoring-target subject images IM, scope-related information, and processor-related information in the form of a database. Then, in step S503, the controller 220 performs image analysis on a monitoring-target subject image IM that has been received. Specifically, the controller 220 detects the degree of a color shift of the monitoring-target subject image IM from the color white, based on the ratio between the levels of R, G, and B color pixel signals.

FIG. 10 shows a monitoring-target subject image IM that has a normal color tone, and a monitoring-target subject image IM″ that has a color tone that is out of an allowable range. If a signal cable or an optical fiber that transmits image signals output from the image sensor 12 in the video scope 10 to the processor 30 has deteriorated, perfect (ideal) white cannot be acquired through white balance adjustment processing. This is because gain adjustment through white balance adjustment processing has a limitation, and it is impossible to address changes in characteristics of the image sensor 12 and the signal cable by adjusting the gain values of R, G, and B color image signals through white balance adjustment processing.

In step S504, the controller 220 determines whether or not the ratio between the values of the R, G, and B color image signals of the monitoring-target subject image IM is out of an allowable range (e.g. the average value of the R pixel signals is greater than the average value of the G and B pixel signals by a predetermined value). If the ratio between the values of the R, G, and B color image signals of the monitoring-target subject image IM is out of the allowable range, i.e., if the color tone of the monitoring-target subject image IM is out of the allowable range, the controller 220 displays a warning on the monitor 400, and transmits, to the target endoscope device 100, a warning command indicating that the endoscope device 100 requires maintenance, via the communication unit 212 (S505 and S506). The endoscope device 100 that has received the warning command performs the same warning display processing as in the second embodiment (see FIG. 8).

In this way, according to the third embodiment, the controller 220 performs image analysis in conjunction with filing of the monitoring-target subject images IM, and determines whether or not the endoscope device 100 requires maintenance. The controller 220 can determine whether or not the endoscope device 100 requires maintenance by only analyzing the monitoring-target subject images IM that have been received, without creating a chronological database from the monitoring-target subject images IM. Also, even if unexpected image deterioration (such as halation) occurs in a monitoring-target subject image IM, the filing device 200 can address the deterioration at the time of filing.

Note that image analysis may be performed in the second embodiment as in the third embodiment. That is, the controller 220 may be configured to calculate the proportion of pixels that express noise, to all of the pixels of the monitoring-target subject image IM, and determine that the endoscope device 100 requires maintenance when the proportion is no less than a threshold value. For example, the monitoring-target subject image IM″ shown in FIG. 10 may contain line noise or dot noise. Such noise is likely to occur due to deterioration of a flexible thin cable that extends in the scope leading end part 10T of the video scope 10. The endoscope device 100 may be configured to determine that the endoscope device 100 requires emergency maintenance if the result of the image analysis shows that the color tone of the image has changed as in the monitoring-target subject image IM″ in FIG. 10 and the proportion of pixels that express noise is no less than a predetermined threshold value.

The monitoring-target subject image IM used in the first to third embodiment is an image that has just undergone white balance adjustment processing. However, the monitoring-target subject image IM is not necessarily such an image. The monitoring-target subject image IM may be an image in which, although white balance adjustment processing has been started, the gains of the R, G, and B color image signals have not been adjusted, or an image at the time when a predetermined period of time has elapsed since the completion of white balance adjustment processing.

In addition, the endoscope management system according to the present embodiment does not necessarily include a plurality of endoscope devices 100, and may be configured such that a single endoscope device 100 is connected to the filing device 200.

### DESCRIPTION OF REFERENCE SIGNS

10 Video Scope
11 Light Guide
11A Incident End
12 Image Sensor
30 Processor
60 Monitor
100 Endoscope Device
200 Filing Device (Management Device)
250 Computer
300 Keyboard
400 Monitor

1. An endoscope management system comprising:
at least one endoscope device that includes a video scope and a processor, the video scope being configured to capture an image of a subject, and the processor including an image signal processing unit configured to execute white balance adjustment processing, using an image of the subject captured by the video scope; and
a management device configured to be connected to the processor of the endoscope device such that the management device is able to perform mutual communication with the processor,
wherein the endoscope device includes a control unit configured to transmit, in response to the execution of the white balance adjustment processing, monitoring-target subject images acquired through the white balance adjustment processing, to the management device, and
the management device includes a memory configured to record the monitoring-target subject images, and a main management unit configured to manage deterioration of the endoscope device, using the monitoring-target subject images thus recorded.

2. An endoscope management system comprising:
at least one endoscope device that includes a white balance adjustment processing unit that executes white balance adjustment processing according to an operation performed by an operator; and
a management device that is connected to the endoscope device such that the management device is able to perform mutual communication with the endoscope device,
wherein the endoscope device transmits, in response to the execution of the white balance adjustment processing, monitoring-target subject images acquired through the white balance adjustment processing, to the management device, and
the management device records the monitoring-target subject images thus received, in a memory.

3. The endoscope management system according to claim 2,
wherein the endoscope device includes a video scope and a processor, the video scope being configured to capture an image of a subject, and the processor including an image signal processing unit including the white balance adjustment processing unit, the white balance adjustment processing unit being configured to execute white balance adjustment processing, using images of the subject captured by the video scope as the monitoring-target subject images.

4. The endoscope management system according to claim 1,
wherein the endoscope device transmits scope-related information related to the video scope of the endoscope device, in conjunction with transmission of the monitoring-target subject images.

5. The endoscope management system according to claim 4,
wherein the scope-related information includes information regarding a usage history of the video scope and identification information of the video scope.

6. The endoscope management system according to claim 5,
wherein information regarding the usage history of the video scope includes a cumulative scope-energization time that indicates a cumulative time for which the video scope has been energized, or a scope connection count that indicates a number of times the video scope has been connected to the processor.

7. The endoscope management system according to claim 1,
wherein the endoscope device transmits processor-related information related to the processor of the endoscope device, in conjunction with transmission of the monitoring-target subject images.

8. The endoscope management system according claim 1,
wherein the processor includes a processing circuit that is able to turn ON and OFF image processing that is to be performed on images of the subject captured by the video scope, and
the processing circuit turns the image processing OFF when the white balance adjustment processing is to be performed.

9. The endoscope management system according to claim 1,
wherein the endoscope device transmits scope-related information related to the video scope and processor-related information related to the processor to the management device together with the monitoring-target subject images, and
the main management unit associates the plurality of monitoring-target subject images with different dates recorded in the memory, with at least one of the scope-related information and the processor-related information transmitted in conjunction with each of the plurality of monitoring-target subject images, to determine whether or not the endoscope device requires maintenance.

10. The endoscope management system according to claim 9,
wherein the scope-related information includes information regarding cumulative scope-energization time that indicates cumulative time for which the video scope has been energized, and
the management device determines that the endoscope device requires maintenance when the cumulative scope-energization time is no less than a predetermined period of time.

11. The endoscope management system according to claim 9,
wherein the management device determines whether or not the endoscope device requires maintenance based on image analysis performed on the monitoring-target subject images recorded in the memory.

12. The endoscope management system according to claim 11,
wherein the management device determines that the endoscope requires maintenance when a color tone of any of the monitoring-target subject images is out of an allowable range.

13. The endoscope management system according to claim 10,
wherein the management device notifies the endoscope device of the result of the determination regarding whether or not the endoscope device requires maintenance.

14. The endoscope management system according to claim 1,
wherein the monitoring-target subject images are images of a tubular inner surface of a white balance adjustment tool, and are color still images that have undergone white balance adjustment processing.

15. The endoscope management system according to claim 1,
wherein the management device is connected to a monitor that is controlled such that the monitor displays the monitoring-target subject images together with at least one of scope-related information related to the video scope of the endoscope device and processor-related information related to the processor.

16. An endoscope device comprising:
a video scope configured to capture an image of a subject; and
a processor including an image signal processing unit and a control unit, the image signal processing unit being configured to execute white balance adjustment processing, using images of the subject captured by the video scope, and the control unit being configured to transmit, in response to the execution of the white balance adjustment processing, monitoring-target subject images acquired through the white balance adjustment processing, to a management device, which is an external device.

17. A management device configured to be connected to the endoscope device according to claim 16 such that the management device is able to perform mutual communication with the endoscope device, the management device comprising:
a communication unit configured to receive the monitoring-target subject images output from the endoscope device; a memory configured to record the monitoring-target subject images thus received; and a main management unit configured to manage deterioration of the endoscope device, using the monitoring-target subject images thus recorded.

18. A method for managing an endoscope device,
wherein at least one endoscope device that includes an image signal processing unit configured to execute white balance adjustment processing is connected to a management device such that the end scope device is able to perform mutual communication with the management device, and
the method comprises:
transmitting, using the endoscope device, in response to the execution of the white balance adjustment processing, a monitoring-target subject image acquired through the white balance adjustment processing, to the management device;
recording, using the management device, the monitoring-target subject images thus received, in a memory; and
managing, using the management device, deterioration of the endoscope device, using the monitoring-target subject images thus recorded.
